(19) United States
(12) Patent Application Publication
(10) Pub. No.: US 2004/0116523 A1
(43) Pub. Date: Jun. 17, 2004

(54) Use of c4-c10 acids for preventing gram-negative bacterial infections

(76) Inventor: Michel Yvan Popoff, Plaisir (FR)

Correspondence Address: MUSERLIAN AND LUCAS AND MERCANTI, LLP, 600 THIRD AVENUE, NEW YORK, NY, 10016, US

(21) Appl. No.: 10/312,047

(22) PCT Filed: Jun. 22, 2001
(86) PCT No.: PCT/FR01/01971

(30) Foreign Application Priority Data
Jun. 22, 2000 (FR) 00/07992
Jun. 29, 2000 (FR) 00/08383

(51) Int. Cl. A61K 31/20

(52) U.S. Cl. 514/558; 514/559

(57) ABSTRACT

The invention relates to the use of C₄-C₁₀ acids and/or of at least one of their salts or esters, for preparing a pharmaceutical composition intended to prevent Gram-negative bacterial infections, in particular Salmonella infections.

The present invention relates to the use of C₄-C₁₀ acids for preventing Gram-negative bacterial infections, in particular Salmonella infections, both in animals and in humans.

The Salmonellae are enteroinvasive bacteria which are pathogenic for humans and animals. The crucial step in the triggering of salmonellosis, whether it involves gastroenteritis or a generalized infection of typhoid fever type, is the entry of the Salmonellae into the ileal epithelial cells.

It has now been thoroughly demonstrated that the majority of the genes required for this entry (invasion genes) are grouped together on the SPI-1 pathogenicity island at centisome 63 on the Salmonella chromosome (J. E. Galan, Mol. Microbiol., 1996, 20, 263-271). These genes encode the IagA (or HilA) transcriptional activator, belonging to the OmpR/ToxR family, for the components of the inv-spa-prg type III secretion apparatus and for its targets including the SipBCDA (or SspBCDA), StpA (or SptP) and AvrA proteins. The expression of the iagA gene is, itself, very tightly controlled.

The transcription of iagA requires the production of two derepressors, SprA (or HilC or SirC) and HilD, which are encoded by SPI-1 genes and form part of the AraC/XylS family (Eichelberg et al., 1999, Mol. Microbiol., 1999, 33, 139-152; Schechter et al., Mol. Microbiol., 1999, 32, 629-642). The transcription of iagA is also controlled directly or indirectly by two-component systems, RcsB-RcsC and PhoP-PhoQ, which are not encoded by genes located at centisome 63. The RcsB-RcsC system responds to osmolarity (Arricau et al., Mol. Microbiol., 1998, 29, 835-850) and the PhoP-PhoQ system responds to the concentration of divalent cations, such as calcium and magnesium ions (Garcia Vescoci et al., Cell, 1996, 84, 165-174; Miller et al., Proc. Natl. Acad. Sci. USA, 1989, 86, 5054-5058) of the environmental medium.

Other environmental conditions (low oxygen tension, slightly alkaline pH) are also required for the expression of iagA; however, the mechanisms involved have not been identified.

When the osmolarity and the concentration of calcium or magnesium ions are low (in water for example), the PhoP-PhoQ and RcsB-RcsC systems repress directly or indirectly the expression of iagA. In the absence of the IagA activator, the genes encoding the components and targets of the Inv-Spa-Prg secretion apparatus are not expressed, and the Salmonellae are incapable of entering into epithelial cells in culture.

On the other hand, when the osmolarity and the concentration of calcium or magnesium ions are high (in the intestinal lumen for example), the PhoP-PhoQ and RcsB-RcsC systems no longer repress the expression of iagA.

It is presumed that the SprA and HilD derepressors, by binding to the iagA promoter or by interfering with the PhoP or RcsB regulators, are involved in this regulation mechanism. The IagA protein will then activate the transcription of the inv, spa and prg operons. The product of invf, the first gene of the inv operon, is, itself, a regulator of the AraC family and will activate the transcription of the sip operon (Kaniga et al., Mol. Microbiol., 1994, 13, 555-568). At this stage, the components of the Inv-Spa-Prg secretion apparatus are produced. Its targets, in particular the Sip proteins, are also synthesized, but they remain stored in the cytoplasmic compartment since the secretion apparatus is inactive. The activity of the secretion apparatus will be triggered by contact with the epithelial cell (Galan, 1996). The Sip proteins will be secreted and will form a translocator, which will be used to inject the effector proteins, including StpA and AvrA, into the cytosol of the target cell.

These effector proteins then activate various signalling pathways, causing a variety of cellular responses and, finally, the entry or the Salmonellae into the cell. However, it has been shown that these two conditions (osmclarity and high concentration of divalent cations) must be simultaneously optimal in order for the Salmonellae to fully express their invasive capacity.

If just one of these conditions is modified, the invasive capcapacity of the Salmonellae is then very greatly decreased (Balaj et al., Mol. Miczobiol.. 1996, 22, 703-714). For this reason, the Salmonellae cultured in a medium with a low osmolarity or with a low concentration of divalent cations are incapable of penetrating into epithelial cells in culture.

The set of data reported above shows that the expression of the entry phenotype in Salmonella is very finely controlled by regulators acting in cascade (probably not all are identified) as a function of the environmental conditions.

If just one of these regulators is interfered with, then a noninvasive phenotype must result therefrom.

While it is very easy to vary the osmolarity or concentration of divalent cations of a cell culture medium in vitro, for the purpose of blocking the invasion of Salmonellae into these epithelial cells in culture, it is not, however, possible to carry out the same approach in vivo.

During the last fifteen years, the number of Salmonella infections has considerably increased. Each year throughout the world typhoid fever is responsible for 600 000 deaths for approximately 20 million cases.

In France, salmonellosis caused by S. enteritidis represents approximately 33% of cases of toxic food poisoning.

The Salmonellae therefore still constitute a major public health problem. Epidemiological studies have clearly shown that this upsurge in salmonelloses (except in the case of typhoid fever, which is a strictly human disease) is due to the consumption of products of animal origin which are contaminated by Salmonella. One of the most demonstrative examples is that of S. enteritidis, which has been the cause of a worldwide epidemic. The source of contamination by S. enteritidis has been completely identified. It is eggs and egg-based products.

The situation risks becoming even more preoccupying with the generalization of the use of antibiotics in animal feed. The consequence of this farming practice is the appearance of bacterial strains which are multiresistant to antibiotics, to such an extent that the panoply of the various antibiotics available may very rapidly become insufficient.

This phenomenon has, moreover, led to drastic measures being taken in certain cases. For example, for poultry farms, the European directive 92/117 imposes on Member States a standardized monitoring of the presence of Salmonella in breeding flocks. Point V of Annex III specifies that flocks which are recognized to be contaminated by S. typhimurium or S. enteritidis must be slaughtered. This provision has recently been modified by directive 97/22, which makes it possible to continue to exploit flocks contaminated by S. typhimurium or S. enteritidis if it is established, to the satisfaction of the competent authority, that the infection due to these two bacteria has disappeared.

Many investigations have been related to the study of the bacteriostatic or bactericidal activity of various acylated monoglycerides and fatty acids.

Thus, it has been shown (Petschow et al., J. Med. Microbiol. 1998, 47, 383-389) that lauric acid for example, which is a saturated fatty acid in which the chain is formed by 12 carbon atoms, exhibits bactericidal activity in vitro on S. typhi, Vibrio cholerae and Shigella sonnei at the concentration of 0.25 g/l.

According to this same article, it appears that, at the concentration of 1.25 g/l, caprylic acid (CH₃(CH₂)₆COOH), which is a short-chain (8 carbon atoms) saturated fatty acid, has no bactericidal activity in vitro on S. typhi, Vibrio cholerae, enteropathogenic and enterotoxigenic E. coli and Shigella sonnei.

It has also been shown, in vitro, that, at the concentration of 7.8 mM, caprylic acid has no bacteriostatic or bactericidal effect on Gram-positive or Gram-negative bacteria, whereas other fatty acids with a longer, in particular from 12 carbon atoms upwards, saturated carbon-based chain, such as lauric acid, or some unsaturated fatty acids, such as for example linoleic acid and oleic acid, are very effective (Kabara et al., Antimicrob. Agents Chemother., 1972, 2, 23-31).

Finally, many authors have demonstrated that certain fatty acids, such as for example n-hexanoic acid, caprylic acid and decanoic acid, have, at acid PH, bactericidal activity on a great variety of Gram-positive or Gram-negative microorganisms and make it possible to treat various types of infection (US patents 4 489 097 and 4 406 884, patent application EP 0 021 504). Those documents specify, however, that, in order to be effective, these acids must be used at acid pH so as to be in free acid form.

It appears, therefore, that the bacteriostatic or bactericidal activity of fatty acids is linked to the length of the carbon-based chain and to the number of unsaturations that it carries, the most significant activity being attributed to fatty acids comprising at least 12 carbon atoms, and preferably one or two unsaturations.

Faced with this major problem, namely Gram-negative bacterial infections and in particular salmonelloses, and knowing that the use of antibiotics in animal feed will very certainly be prohibited in a few years' time, at least in the Member States of Europe, there is a real need to develop compositions which make it possible to prevent Gram-negative bacterial infections, and in particular Salmonella infections, in order to avoid the use of antibiotics and the phenomena of resistance.

It is in order to remedy this problem that, surprisingly, the inventors have developed what forms the subject of the invention.

A subject of the present invention is therefore the use of an effective amount of at least one compound of formula (I) as follows:

R₁-COO-R₂ (I)

in which:

R₁ represents a C₄-C₉ saturated carbon-based chain, optionally substituted with one or more hydroxyl or amine functions, or with an aromatic ring;

R₂ represents a hydrogen atom, a monovalent alkali metal atom or an alkyl radical, it being understood that, when R₂ represents a hydrogen atom and R₁ represents a C₇ saturated carbon-based chain substituted with an amine function, then said amine function is not at position 2 or 8; as an active principal, for preparing a pharmaceutical composition with a neutral pH intended to prevent Gram-negative bacterial infections, and in particular Salmonella infections, both in humans and in animals.

Depending on what the R₂ radical means, the compounds of formula (I) above can, therefore, be present in the form of an acid, of a salt or of an ester.

The inventors have, in particular, demonstrated that the preventive administration of a composition with a neutral pH containing at least sodium caprylate to mice subsequently infected with three major serotypes of Salmonella makes it possible to considerably decrease the level of splenic colonization by these bacteria, even though this composition has no bacterial activity at such a pH.

According to the invention, the pH of the pharmaceutical composition used is preferably between 6.5 and 7.5, and even more preferably between 7.1 and 7.4.

A pH value which is particularly suitable for the use in accordance with the invention is one between 7.2 and 7.3.

Among the compounds of formula (I) above, mention may be made, in particular, of valeric acid, caproic acid, oenanthic acid, caprylic acid and pelargonic acid, and their monohydroxylated derivatives, and also their salts and their esters.

According to a particular embodiment of the invention, and when the R₁ radical of the compounds of formula (I) represents a carbon-based chain substituted with a hydroxyl function, then said hydroxyl function is at position 2 when the R₁ radical contains 4 to 6 carbon atoms and at position 2 or 8 when the R₁ radical contains 7 to 9 carbon atoms. Among these particular compounds of formula (I), mention may in particular be made of 2-hydroxycaprylic acid and 8-hydroxycaprylic acid.

Among the compounds of formula (I), caprylic acid (which corresponds to a compound of formula (I) in which R₁ represents a C₇ saturated carbon-based chain), its derivatives hydroxylated at position 2 or 8, their salts and their esters are particularly preferred.

In fact, besides its antifungal properties (Wyss et al., Arch. Biochem., 1945, 7, 418), caprylic acid used at a neutral pH makes it possible, unexpectedly, to prevent Salmonella infections, even though, at such a pH value, this acid has no bactericidal activity. According to the invention, the alkali metal atoms defined for the R₂ radical are preferably chosen from sodium and potassium.

According to the invention, the alkyl radicals defined for the R₂ radical are preferably chosen from C₁-C₄ alkyl radicals, among which methyl, ethyl and butyl radicals are most particularly preferred.

According to an advantageous embodiment of the invention, use is made of sodium caprylate, i.e. a compound of formula (I) in which R₁ represents a C₇ saturated carbon-based chain and R₂ represents a sodium atom.

The effective amount of compound(s) of formula (I) corresponds preferably to single doses of between 20 mM and 200 mM, and more preferably of between 50 mM and 100 mM.

The pharmaceutical composition used in accordance with the invention can also contain one or more additional active principles.

The compounds of formula (I) used in accordance with the invention can, of course, be formulated in a pharmaceutically acceptable vehicle consisting of one or more excipients conventionally used for preparing pharmaceutical compositions, such as anti-aggregating agents, antioxidants, colorants, vitamins, mineral salts, flavour enhancers, or smoothing, assembling or isolating agents, and, in general, any excipient conventionally used in the pharmaceutical industry.

Of course, those skilled in the art will make sure, in this instance, that the additive(s) optionally used is (are) compatible with the intrinsic properties attached to the present invention; in particular the use of these additives should have no incidence on the pH of the pharmaceutical compositions used.

The pharmaceutical composition used according to the present invention is preferably administered orally, and can be in various forms, such as in the form of tablets, gelatin capsules, drinkable suspensions or lozenges, or in any other form suitable for the oral administration method.

The pharmaceutical composition used according to the present invention can, in particular, be added to the drinking water and/or to the feed distributed to farm-stock animals (poultry, cattle, pigs, sheep, etc.) so as to decrease the incidence of Gram-negative bacterial infections, and in particular Salmonella infections, and limit carrying, and thus reduce the risk of a subsequent contamination of humans.

The pharmaceutical composition used according to the present invention can also be administered to humans as a preventive medicinal product in order to reduce the risk of infection in individuals staying for limited periods in a region highly endemic in particular for salmonelloses.

The pharmaceutical composition used according to the present invention, coupled with mass vaccination, and while awaiting the setting up of immune protection, can also be used in humans to stop or slow down an epidemic of typhoid fever.

Besides the preceding arrangements, the invention also comprises other arrangements which will emerge from the following description, which refers to an example of demonstration of the activity of sodium caprylate in preventing salmonelloses in mice, and also to the attached figures, in which:

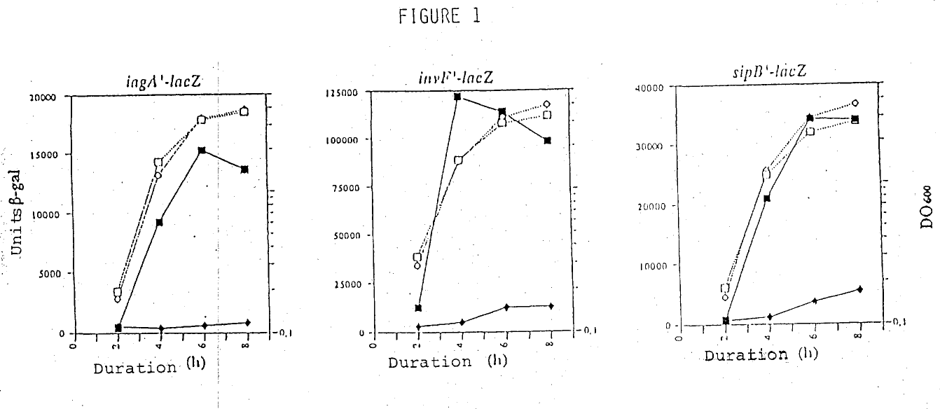

FIG. 1 shows the effect of the concentration of Ca²+ ions on the expression of the iagA′-lacZ, invF′-lacZ and sipB′-lacZ fusions;

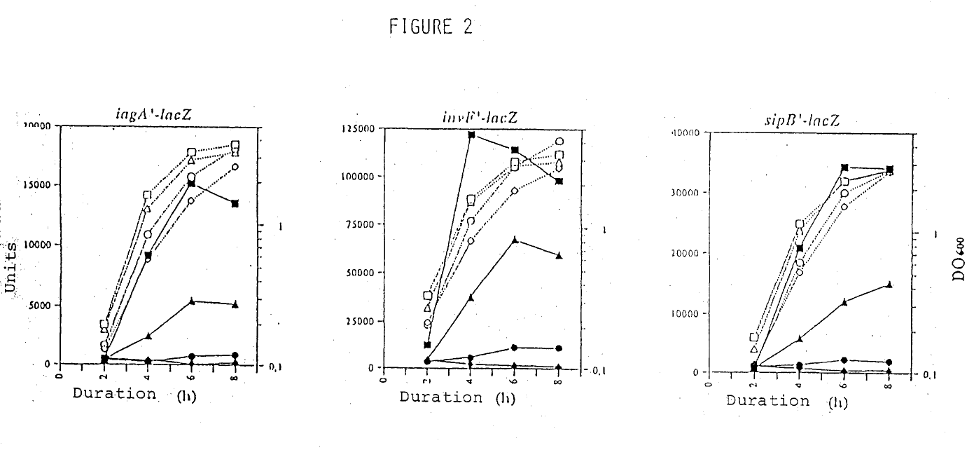

FIG. 2 shows the effect of the concentration of sodium benzoate on the expression of the iagA′-lacZ, invF′-lacZ and sipB′-lacZ fusions;

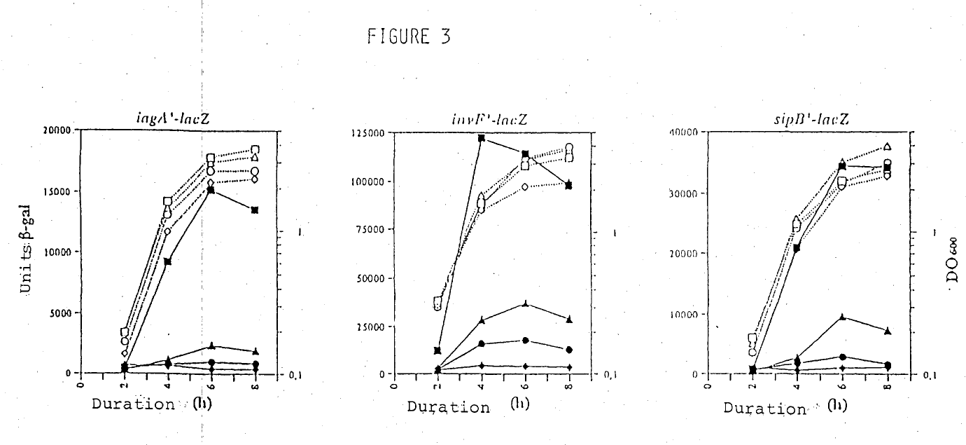

FIG. 3 shows the effect of the concentration of sodium caprylate on the expression of the iagA′-lacZ, invF′-lacZ and sipB′-lacZ fusions;

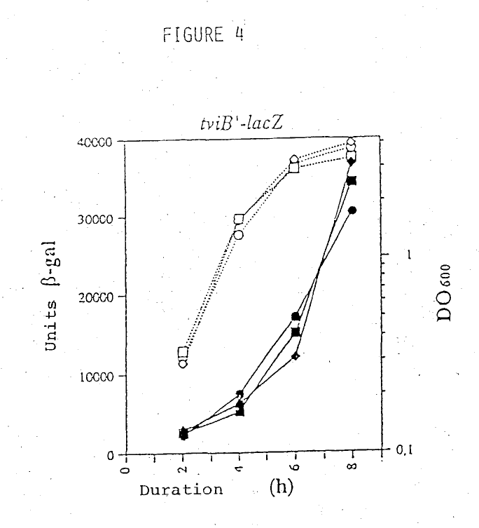

FIG. 4 shows the effect of sodium benzoate and of sodium caprylate on the expression of the tviB′-lacZ fusion;

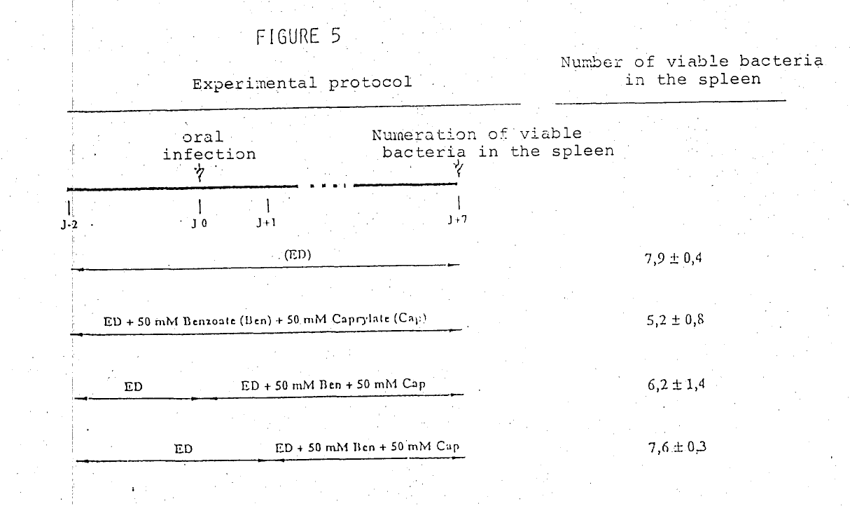

FIG. 5 shows the effect of a mixture of sodium benzoate and of sodium caprylate on the splenic colonization of mice infected orally with S. typhimurium C52.

It should, however, be clearly understood that this example is given only by way of illustration of the subject of the invention, of which it in no way constitutes a limitation.

### EXAMPLE 1 : DEMONSTRATION OF THE PREVENTIVE ACTIVITY OF SODIUM CAPRYLATE ON SALMONELLA INFECTIONS

I) Materials and methods

I-a) Strains, media and culture conditions

The list of Salmonella strains used in this example are shown in Table I hereinafter:

TABLE I

| Strain | Characteristics | Origin or reference |
|---|---|---|
| S. typhi Ty2 | Parental strain | WHO collection |
| | (PS) | Reference Felix 59 |
| S. typhi Ty266 | Ty2 carrying the | Virlogeux et al., |
| | tviB′-lacZcat | Microbiology, 1995, |
| | fusion | 141, 3039-3047 |
| S. typhi Ty267 | Ty2 carrying the | Arricau et al., |
| | iagA′-lacZcat | Molecular |
| | fusion | Microbiology, 1998, |
| | | 29, 835-850 |
| S. typhi Ty272 | Ty2 carrying the | Arricau et al., |
| | invF′-lacZcat | 1998, mentioned |
| | fusion | above |
| S. typhi Ty277 | Ty2 carrying the | Arricau et al., |
| | sipB′-lacZcat | 1998, mentioned |
| | fusion | above |
| S. typhimurium | PS, virulent for | WHO collection |
| C52 | mice | Reference C52 |
| S. dublin 5917 | PS, virulent for | WHO collection |
| | mice | Reference 5917 |
| S. enteritidis | PS, virulent for | Thorns et al., |
| LA5 | mice | Microbial |
| | | Pathogenesis, 1996 |
| | | 20, 235-246 |

The antigenic formula of each Salmonella strain was controlled by slide agglutination with antisera specific for somatic and flagellar antigenic factors, sold by the company BioRad-Diagnostics Pasteur.

Routinely, the strains were cultured at 37° C. on agar or in typto-casein-soya broth (TCS; sold by the company BioRad-Diagnostics Pasteur).

In order to study the expression of the invasion genes in vitro, two media which are derived from standard LB medium (Sambrook et al., Molecular Cloning, 1989) were prepared:

a medium favourable to the expression of the Salmonella invasion genes (favourable LB medium), and

an LB medium unfavourable to the expression of the Salmonella invasion genes (unfavourable LB medium).

The standard LB medium of origin contains 170 rnM of NaCl and 850 ±50 μM of Ca²+ ion.

The favourable LB medium (high osmolarity and concentration of Ca²+ions) was defined as being the standard LB medium modified so as to have a final NaCl concentration of 300 mM and a final Ca concentration of 5 mM.

The unfavourable LB medium (high osmolarity and low concentration of Ca²+ ions) was defined as being the standard LB medium modified so as to have a final NaCl concentration of 300 mM, and is supplemented with 1 mM of ethylene glycol bis(β-aminoethyl ether) N,N,N′,N′-tetraacetic acid (EGTA) so as to create a divalent cation depletion of the medium.

The mobility of each strain was studied using the U-tube culturing technique (Popoff and Le Minor, Guide pour la preparation des serum anti-Salmonella (Guide for preparing salmonella antisera], 1997). When necessary, the culture media were supplemented with chloramphenicol (Cm) at 15 μg/ml.

I-b) Determination of the μ-galactosidase activity

Each of the strains described above was cultured at 37° C. on agar or in TCS broth. The P-galactosidase activity was determined using 4-methyl-umbelliferyl-p-D-galactopyranoside sold by the company Sigma, according to the technique of Klarsfeld et al. (Mol. Mibrobiol., 1994, 13, 585-597). The results, expressed in P-galactosidase units (fluorescence per hour and per OD₆₀₀), represent the mean of the values obtained in at least three independent experiments. The standard deviations were less than 10% of the values presented.

I-c) Use of “Biotype 1001”® plates

A “Biotype 100”® plate sold by the company BioMerieux is composed of 100 microcupules. Each micro-cupule contains a dehydrated carbon-based substrate, with the exception of the first cupule, which is a control without substrate (the amount of dehydrated substrate per cupule is not given by the manufacturer).

Normally, the “Biotype 100”® plate makes it possible to study the nutritional capacity of a bacterial strain on 99 different sources of carbon (auxanogram).

In the context of the present example, these plates were used to search for carbon-based compounds which would block the expression of the invasion genes in Salmonella.

For this, the S. typhi strain Ty267, which is a derivative of the parental strain Ty2 carrying the iagA′-lacZcat transcriptional fusion (Table I), was cultured in TCS broth for 18 hours at 37° C. with shaking (200 rpm).

This culture was then diluted 100-fold in favourable LB medium (0.6 ml of culture for 60 ml of favourable LB), and then this suspension was used immediately to seed the 100 cupules of a “Biotype 100”® plate, in a proportion of 450 μl of suspension per cupule.

After incubation for 36 hours in an incubator at 37° C., the activity of the iagA′-lacZcat fusion in the Ty267 strain was determined for the culture of each cupule.

By comparing the value of the P-galactosidase activity measured in a cupule containing a carbon-based substrate with that of the control cupule without substrate, it was possible to investigate whether a substrate had no effect on, activated or repressed the expression of the iagA′-lacZcat fusion.

I-d) Experimental infection of mice

The 5-6-week-old female C57Bl/6 mice used in the context of this experiment originated from the IFFA-CREDO breeding centre (LArbresle, France).

Throughout the duration of the experiments, the mice had access ad libidum to the drinking water which had a given composition for each experiment.

They were infected orally in the following way. The Salmonella strains were cultured in TCS broth for 18 hours at 37° C. with shaking (200 rpm). This culture was then adjusted to 108 bacteria/ml (in the stationary growth phase, 1 OD₆₀₀ =1.5 ×109 bacteria/ml), and then the number of viable bacteria was controlled by counting on TCS agar.

On the day of infection (DO), the drinking-water bottles of the mice were replaced with bottles containing 100 ml of drinking water to which the required number of bacteria had been added.

After infection for 24 hours (D++1), the bottles containing the contaminated drinking water were replaced with bottles containing uncontaminated drinking water.

Seven days after infection (D+7), the spleens of the mice infected in this way were removed and homogenized separately in 1 ml of physiological saline containing 0.7% of NaCl.

The number of viable bacteria per spleen was determined by plating out dilutions of the homogenate, onto TCS agar.

The effect of the composition of the drinking water on the splenic colonization by Salmonella was estimated using the Student's t test.

For the survival experiments, the mice were infected as indicated above and the mortality of the animals was recorded for 21 days.

I-e) Nature of the acids tested

In addition to the caprylic acid, aromatic acids were studied for the purpose of comparing their properties to those of the caprylic acid. The aromatic acids studied were as follows: benzoic acid, phenylacetic acid, 3-phenylpropionic acid and 4-phenylbutyric acid.

The aromatic acids and the caprylic acid originate from the company Sigma. They were all used in the form of their sodium salt and at a pH of between 7.2 and 7.3.

II) Results

II-a) Preliminary step: effect of the concentration of divalent cations on the expression of the Salmonella invasion genes

The purpose of this preliminary step was to demonstrate that lacZ transcriptional fusions could be used to investigate the environmental conditions which repress the expression of the genes present on the SPI-1 pathogenicity island.

For this, the S. typhi strains Ty267, Ty272 and Ty277, which carry the iagA′-lacZ, invF′-lacZ and sipB′-lacZ fusions, respectively, (see Table I), were cultured in the favourable LB or unfavourable LB medium.

The β-galactosidase activity expressed by the fusions was measured at different times of culturing. The results appear in FIG. 1. Samples were taken every two hours in order to measure the OD₆₀₀ (open symbols on FIG. 1) and in order to determine the β-galactosidase activity (solid symbols on FIG. 1).

The S. typhi strains Ty267 (iagA′-lacZ), Ty272 (invF′-lacZ) and Ty277 (sipB′-lacZ) were cultured in LB medium containing 300 mM NaCl and 5 mM of CaCl₂ (represented with a square) or 300 mM NaCl and 1 mM EGTA (represented with a diamond).

These results show that the expression of the fusions is very greatly decreased when the culture medium is depleted of divalent cations.

They consequently confirm that it is sufficient to modify a single favourable condition for the invasion genes to no longer be expressed in S. typhi, as had been demonstrated in S. typhimurium (Bajaj et al., 1996, mentioned above).

Consequently, the iagA′-lacZ, invF′-lacZ and sipB′-lacZ transcriptional fusions in S. typhi were used in the remainder of this example.

II-b) Effect of certain aromatic acids and of certain fatty acids on the expression of the invasion genes

The S. typhi strain Ty267 (iagA′-lacZ fusion), suspended in the favourable LB medium, was used to seed the “Biotype 100”® plates.

After incubation for 36 hours in an incubator at 37° C., a clear culture was observed in all the cupules, which indicates that none of the 99 carbon-based substrates of the plate has bactericidal or bacteriostatic activity for the Ty267 strain.

The β-galactosidase activity expressed by the iagA′-lacZ fusion was determined for the culture in each cupule. With respect to the β-galactosidase activity measured in the control cupule without substrate (11 250 β-galactosidase units), no substrate caused an increase in the expression of the iagA′-lacZ fusion.

On the contrary, the expression of this fusion was very strongly repressed in the cupules containing the sodium phenylacetate (81 β-galactosidase units), the sodium benzoate (245 β-galactosidase units), the sodium 3-phenylpropionate (166 β-galactosidase units) and the sodium caprylate (47 β-galactosidase units).

Since the amount of substrate contained in each cupule was not known, a more detailed study of the action of these substrates on the expression of the invasion genes was undertaken.

Initially, sodium benzoate and sodium caprylate were used.

The effect of the sodium benzoate and that of the sodium caprylate on the expression of the three fusions is given in FIGS. 2 and 3, respectively.

On these FIGS. 2 and 3, the OD₆₀₀ measurement corresponds to the open symbols and the β-galactosidase activity determination corresponds to the solid symbols.

The S. typhi strains Ty267, Ty272 and Ty277 were seeded in favourable LB medium and in favourable LB medium containing various concentrations of sodium benzoate or of sodium caprylate.

On FIG. 2, the symbols in the form of squares correspond to the favourable LB medium without the addition of sodium benzoate, the symbols in the form of diamonds correspond to the favourable LB medium with the addition of 5 mM of sodium benzoate, the symbols in the form of circles correspond to the favourable LB medium with the addition of 2.5 mM of sodium benzoate and the symbols in the form of triangles correspond to the favourable LB medium with the addition of 1 mM of sodium benzoate.

On FIG. 3, the symbols in the form of squares correspond to the favourable LB medium without the addition of sodium caprylate, the symbols in the form of diamonds correspond to the favourable LB medium with the addition of 5 mM of sodium caprylate, the symbols in the form of circles correspond to the favourable LB medium with the addition of 2.5 mM of sodium caprylate and the symbols in the form of triangles correspond to the favourable LB medium with the addition of 1 mM of sodium caprylate.

The action of these two compounds on the expression of the iagA′-lacZ, invF′-lacZ and sipB′-lacZ fusions was studied by measuring the β-galactosidase activity expressed by these fusions as a function of the time of culturing (samples removed every two hours).

These results show that the presence of one or the other of these two compounds at the final concentration of 5 mM in the culture medium did not significantly affect the growth of the S. typhi strains.

At a concentration greater than or equal to 2.5 mM, the sodium benzoate or the sodium caprylate very strongly represses the expression of the iagA, invf and sipB genes.

When the favourable LB medium contains 1 mM of sodium benzoate, the activity of the fusions is again decreased, by 50% with respect to the values measured in the favourable LB medium without sodium benzoate.

If the favourable LB medium contains 1 mM of sodium caprylate, the β-galactosidase activity expressed by the fusions remains low, of the order of 20% of that measured in the favourable LB medium without sodium caprylate.

If the favourable LB medium is supplemented with 1 mM of sodium benzoate and with 1 mM of sodium caprylate, the activity of the iagA′-lacZ fusion measured after culturing for 6 hours is very low (317 ±12 β-galactosidase units).

This set of results shows that sodium benzoate or sodium caprylate added at the final concentration of 2.5 mM to the favourable LB medium very strongly represses the expression of the Salmonella invasion genes.

These results also suggest that the inhibitory action of the sodium caprylate is greater than that of the sodium benzoate, at least when these products are used at low concentration (1 mM), but that the effect of these two products is additive.

II-c) Effects of sodium benzoate and of sodium caprylate on the specific repression of the expression of the invasion genes of the SPI-1 pathogenicity island

In order to examine whether the effect of the sodium benzoate and of the sodium caprylate was specific for the invasion genes of the SPI-1 pathogenicity island, the action of these two products was studied on the tviB′-lacZ transcriptional fusion, in the S. typhi strain Ty266 (Table I).

The tviB gene has been characterized in S. typhi, and it encodes a GDP-dehydrogenase involved in the biosynthesis of the Vi antigen, which is the capsular polysaccharide of the typhus bacillus (Waxin et al., Res. Microbiol., 1993, 144, 363-371).

The results are reported on FIG. 4, in which it is seen that samples were taken every two hours in order to measure the OD6₀₀ (corresponding to the open symbols) and in order to determine the β-galactosidase activity (corresponding to the solid symbols). The strains were cultured in favourable LB medium without the addition of sodium benzoate or of sodium caprylate (symbols in the form of squares on FIG. 4) and in favourable LB medium containing 5 mM of sodium benzoate (symbols in the form of diamonds on FIG. 4) or 5 mM of sodium caprylate (symbols in the form of circles on FIG. 4).

As shown in FIG. 4, the sodium benzoate and the sodium caprylate at 5 mM have no significant effect on the expression of the tviB′-lacZ fusion.

Several components of the Inv-Spa-Prg secretion apparatus show similarities with components of the machinery involved in the secretion of flagellin and the assembly of flagella.

In fact, it has been shown in S. typhi that the secretion of the Sip proteins, the amount of flagellin secreted and the mobility are regulated in a coordinated way in response to the osmolarity of the culture medium (Arricau et al., 1998, mentioned above).

These data led the inventors to study the effect of sodium benzoate and of sodium caprylate on the mobility of the S. typhi strain Ty2.

This study showed that the Ty2 strain had the same mobility after culturing in a U tube and in a U tube supplemented with 5 mM of sodium benzoate or with 5 mM of sodium caprylate.

It results therefrom that sodium benzoate and sodium caprylate specifically repress the expression of the invasion genes of the SPI-l pathogenicity island in Salmonella.

II-d) Effects of certain aromatic acids on the expression of the invasion genes The S. typhi strain Ty267 (iagA′-lacZ fusion) was seeded in favourable LB medium and in favourable LB medium containing the aromatic acid to be tested at the final concentration of 5 mM.

The β-galactosidase activity of the iagA′-lacZ fusion was measured after incubation for 6 hours at 37° C. with shaking (200 rpm).

As expected according to the results obtained above in “Biotype 100”® ( plates, and as appears in Table II hereinafter, the sodium benzoate, the sodium phenylacetate and the sodium 3-phenylpropionate inhibit the expression of the iagA′-lacZ fusion in S. typhi.

In addition, the β-galactosidase activity of this fusion is also inhibited by the sodium 4-phenylbutyrate (Table II), a substrate not appearing in the “Biotype 100”® plate used above.

TABLE II

| | β-galactosidase activity of |
|---|---|
| Aromatic acid tested (5 mM) | the iagA′-lacZ fusion |
| None (control favourable LB | 12134 ± 1811 |
| medium) | |
| Sodium benzoate | 190 ± 17 |
| Sodium phenylacetate | 149 ± 24 |
| Sodium 3-phenylpropionate | 297 ± 95 |
| Sodium 4-phenylbutyrate | 198 ± 32 |

These results show that the inhibitory activity of these 4 aromatic acids is very similar.

The effect of the sodium benzoate, of the sodium phenylacetate, of the sodium 3-phenylpropionate and of the sodium 4-phenylbutyrate is additive since, if each one of them is added to the favourable LB medium, at the final concentration of 1 mM, the activity of the iagA′-lacZ fusion is strongly repressed (236 ±10 β-galactosidase units), whereas this fusion is only partially repressed when the favourable LB medium is supplemented with 1 mM sodium benzoate alone (5 455 ±223 β-galactosidase units).

II-e) Compared effects of sodium benzoate and of sodium caprylate in a model of murine infection with S. typhimurium C52

The S. typhimurium strain C52 was used to infect C57Bl/6 mice orally.

In this experimental model, the 50% lethal dose (LD₅₀) of the C52 strain is equal to approximately 4 ×10⁵ bacteria (Coynault et al., Mol. Microbiol., 1996, 22, 149-160) and the kinetics of colonization of the spleen have been reported previously (Pardon et al., Ann. Inst. Pasteur/Microbiol., 1986, 137B, 47-60).

Since sodium benzoate and sodium caprylate repress, in vitro, the expression of the Salmonella invasion genes, it was logical to study the effect of these products on the splenic colonization of C57Bl/6 mice infected orally with S. typhimurium C52, with 10⁸ bacteria (250 LD₅₀).

The results are given in FIG. 5. On this figure, the number of viable bacteria is given as the mean (log₁₀) of the values ± the standard deviation. Groups of 8 mice were infected orally with 10⁸ viable bacteria (250 LD₅₀), and the number of viable bacteria was then determined seven days after infection.

These results show that the bacterial load in the spleen is decreased in a highly significant way (p <0.001) in the mice having received drinking water with a pH of between 7.2 and 7.3 containing a mixture of 50 mm of sodium benzoate and 50 mM of sodium caprylate, on the condition that the treatment is initiated two days before infection.

The administration of sodium benzoate and of sodium caprylate on the day of or on the day after infection has no significant effect on the level of splenic colonization.

The effects of the sodium benzoate and of the sodium caprylate, administered, in the drinking water, to the mice two days before infection with S. typhimurium C52, were then studied separately.

The effect of EDTA, which, like EGTA, chelates divalent cations, was studied jointly with the effect of the sodium benzoate or of the sodium caprylate, during murine infection with S. typhimurium.

Groups of five C57Bl/6 mice were infected orally with 5 ×10⁷ bacteria (125 LD₅₀), according to the protocol described above. The results obtained are given in Table III hereinafter:

TABLE III

| Composition of the drinking | Number of viable bacteria |
|---|---|
| water | in the spleen (mean log₁₀) |
| Distilled water (DW) | 7.9 ± 0.3 |
| DW + 50 mM of sodium | 4.5 ± 0.9 |
| benzoate + 50 mM of sodium | |
| caprylate | |
| DW + 50 mM of sodium | 7.8 ± 0.8 |
| benzoate | |
| DW + 50 mM of sodium | 4.3 ± 0.4 |
| caprylate | |
| DW + 50 mM of sodium | 7.9 ± 0.4 |
| benzoate + 10 mM of EDTA | |
| DW + 50 mM of sodium | 6.7 ± 0.9 |
| caprylate + 10 mM of EDTA | |

All these compositions have a pH of between 7.2 and 7.3.

These results show that the administration, two days before infection and for the duration of the experiment, of drinking water containing 50 mM of sodium caprylate decreases by a factor of approximately 1 000 the bacterial load in the spleen on the seventh day of infection.

The administration of a mixture of 50 mM of sodium caprylate and 50 mM of sodium benzoate gives a very comparable result, which indicates that the sodium benzoate does not act in synergy with the sodium caprylate to prevent the murine infection with S. typhimurium C52. In fact, the presence of 50 mM of sodium benzoate in the drinking water has no effect on the level of splenic colonization of the C57Bl/6 mice infected with S. typhimurium C52.

The latter result should be compared to that which had been obtained above, in vitro, on the expression of the iagA′-lacZ, invF′-lacZ and sipB′-lacZ fusions in S. typhi in the presence of sodium benzoate (FIG. 2).

As reported in the preliminary experiments (FIG. 1), the consequence of depleting the favourable medium of divalent cations with EGTA is the repression of the iagA′-lacZ, invF′-lacZ and sipB′-lacZ fusions in S. typhi.

The addition of 10 mM of EDTA to the drinking water containing 50 mM of sodium benzoate confers no protection against the infection with S. typhimurium C52 (Table III).

On the contrary, the presence of 10 mM of EDTA in the drinking water containing 50 mM of sodium caprylate results in a decrease in the protective effect observed with the sodium caprylate alone.

In the same way, the effect of the concentration of sodium caprylate in the drinking water was then studied as a function of the infectious dose.

The results are given in Table IV hereinafter.

TABLE IV

Number of viable bacteria in the
spleen (mean log₁₀)

| Composition of the | Infectious dose | Infectious dose |
|---|---|---|
| drinking water | 5 × 10⁷ bacteria | 10⁷ bacteria |
| Distilled water (DW) | 7.7 ± 0.3 | 7.2 ± 0.3 |
| DW + 50 mM of sodium | 4.6 ± 0.3 | 2.3 ± 0.4 |
| caprylate | | |
| DW + 10 mM of sodium | 6.5 ± 0.4 | 6.5 ± 1.4 |
| caprylate | | |
| DW + 5 mM of sodium | 6.9 ± 0.5 | 6.8 ± 0.2 |
| caprylate | | |

All these compositions have a pH of between 7.2 and 7.3.

These results confirm that the administration, two days before infection and for the duration of the experiment, of drinking water containing 50 mM of sodium caprylate decreases in a very highly significant way the bacterial load in the spleen of the mice infected orally with 5 ×10⁷ bacteria (125 LD₅₀).

In addition, these results show that the addition of 50 mM of sodium caprylate to the drinking water decreases by a factor of approximately 100 000 the bacterial load in the spleen of the mice infected orally with 10⁷ bacteria (25 LD₅₀) On the other hand, the sodium caprylate no longer has a significant protective effect when the concentration thereof is lowered to 5 or 10 mM in the drinking water, whatever the infectious dose used.

II-f) Effect of sodium caprylate on the survival of mice infected with S. typhimurium The effect of sodium caprylate on the survival of mice infected with S. typhimurium C52 was then studied.

A first batch of ten C57Bl/6 mice, to which drinking water, with a pH of between 7.2 and 7.3, without sodium caprylate was administered, was infected orally with 10⁷ bacteria (25 LD₅₀) . No animal survived (3 deaths on the eighth day after infection, 4 on -the ninth and 3 on the tenth).

The second batch of 10 mice, to which drinking water with a pH of between 7.2 and 7.3 containing 50 mM of sodium caprylate had been administered two days before infection and for the duration of the experiment, was also infected orally with 10⁷ bacteria (25 LD₅₀) . One mouse died on the twelfth day of infection. On the twentieth day of infection, the nine remaining mice were sacrificed and the number of bacteria in the spleen was determined. In the spleen of one mouse, log₁₀ =7.3 viable bacteria were counted. The mean (± standard deviation) of the number of viable bacteria in the spleen of the other eight animals was calculated to be equal to log₁₀ =3.3 ±0.3.

These results show that the presence of 50 mM of sodium caprylate in the drinking water with a neutral pH makes it possible to very significantly decrease the bacterial load in the spleen of C57Bl/6 mice infected orally with S. typhimurium. In addition, the sodium caprylate effectively protects the animals against infection with 25 LD₅₀ of S. typhimurium.

II-g) Effect of sodium caprylate in a model of murine infection with S. dublin and S. enteritidis

The S. dublin strain 5917 (Coynault & Norel, Microb. Pathog., 1999, 26, 299-305) and S. enteritidis strain LAS (Thorns et al., Microb. Pathog., 1996, 20, 235-246) were used to infect C57B1/6 mice orally at the dose of 10⁷ bacteria per animal.

The results reported are given in Table V hereinafter:

TABLE V

| | Composition of | Number of viable |
|---|---|---|
| | the drinking | bacteria in the |
| Strain | water | spleen (mean in log₁₀) |
| S. dublin 5917 | Distilled water | 7.7 ± 0.4 |
| | (DW) | |
| S. dublin 5917 | DW + 50 mM of | 2.1 ± 0.2 |
| | sodium caprylate | |
| S. enteritidis | DW | 7.5 ± 0.3 |
| LA5 | | |
| S. enteritidis | DW + 50 mM of | 3.7 ± 0.4 |
| LA5 | sodium caprylate | |

All the compositions used have a pH of between 7.2 and 7.3.

These results show that the addition of 50 mM of sodium caprylate to the drinking water at neutral pH very significantly decreases the bacterial load in the spleen of the mice infected with S. dublin or with S. enteritidis.

Consequently, the preventive effect of the sodium caprylate is independent of the Salmonella serotype, in a murine model of oral infection.

III) Conclusion

This set of results shows that, although sodium benzoate, sodium phenylacetate, sodium 3-phenylpropionate, sodium 4-phenylbutyrate and sodium caprylate inhibit, in vitro, the expression of the Salmonella invasion genes, even though, moreover, the culturing conditions are optimal for the expression of these genes, none of these compounds has any significant bactericidal or bacteriostatic effect on the Salmonellae, at neutral pH.

On the other hand, in a murine model of oral infection, sodium caprylate (compared to sodium benzoate), added at the concentration of 50 mM to the drinking water at neutral pH, makes it possible to considerably decrease the level of splenic colonization by three major Salmonella serotypes: S. typhimurium, S. enteritidis and S. dublin.

It is important to emphasize, in this respect, that the mice used in these experiments belong to the C57Bl/6 line, which is particularly sensitive to Salmonella infection (Mastroeni et al., Fund. Clin. Immunol., 1994, 2, 83-95).

In addition, it emerges from these experiments that sodium caprylate used at neutral pH very effectively protects the animals against infection with 25 LD₅₀ of S. typhimurium, since nine mice out of ten survived this lethal infectious dose.

On the contrary, sodium benzoate has no protective effect in the same model of infection.

Overall, this set of results shows that it is possible to prevent Gram-negative bacterial infections, such as Salmonella infections, with sodium caprylate.

### EXAMPLE 2: COMPARATIVE STUDY OF THE EFFECT OF CERTAIN FATTY ACIDS ON THE EXPRESSION OF INVASION GENES

This study was carried out according to the protocol described above in Example 1.

The aim of this study is to demonstrate that only the compounds corresponding to formula (I) in accordance with the invention make it possible to inhibit the expression of the iagA′-lacZ fusion in S. typhi.

The S. typhi Ty267 strain (iagA′-lacZ fusion) was seeded into favourable LB medium and into favourable LB medium containing the fatty acid to be tested, in the form of its sodium salt, at the final concentration of 5 mM. The various fatty acids tested are given in Table VI hereinafter:

TABLE VI

| | Fatty acid substrate | Chemical |
|---|---|---|
| Fatty acid tested | for Salmonella | formula |
| Acetic acid (*) | Yes | CH₃—COOH |
| Propionic acid (*) | Yes | CH₃—CH₂—COOH |
| Butyric acid (*) | No | CH₃—(CH₂)₂—COOH |
| Valeric acid (*) | No | CH₃—(CH₂)₃—COOH |
| Caproic acid (*) | No | CH₃—(CH₂)₄—COOH |
| Oenanthic acid (*) | No | CH₃—(CH₂)₅—COOH |
| Caprylic acid (*) | No | CH₃—(CH₂)₆—COOH |
| 2-Hydroxycaprylic | No | CH₃—(CH₂)₅— |
| acid | | CH(OH)—COOH |
| 8-Hydroxycaprylic | No | CH₂OH—(CH₂)₆—COOH |
| acid | | |
| 2-Aminocaprylic | No | CH₃—(CH₂)₅— |
| acid (*) | | CH(NH₂)—COOH |
| 8-Aminocaprylic | No | (NH₂)—CH₂—(CH₂)_{6—} |
| acid (*) | | COOH |
| Pelargonic acid | No | CH₃—(CH₂)₇—COOH |

(*): Compound not in accordance with the invention since it does not correspond to formula (I).

The β-galactosidase activity of the iagA′-lacZ fusion was measured after incubation for 6 hours at 37° C. with shaking (200 rpm).

The results obtained are given in Table VII hereinafter:

TABLE VII

| | β-Galactosidase activity |
|---|---|
| Aromatic acid tested (5 mM) | of the iagA′-lacZ fusion |
| None (control favourable LB | 22022 ± 1177 |
| medium) | |
| Acetic acid (*) | 29265 ± 1692 |
| Propionic acid (*) | 14650 ± 1333 |
| Butyric acid (*) | 21225 ± 1741 |
| Valeric acid (*) | 509 ± 91 |
| Caproic acid (*) | 347 ± 21 |
| Oenanthic acid (*) | 525 ± 43 |
| Caprylic acid | 445 ± 69 |
| 2-Hydroxycaprylic acid | 225 ± 72 |
| 8-Hydroxycaprylic acid | 593 ± 49 |
| 2-Aminocaprylic acid (*) | 33149 ± 2520 |
| 8-Aminocaprylic acid (*) | 18077 ± 1263 |
| Pelargonic acid | 383 ± 54 |

These results show:

that the sodium salts of acetic acid, propionic acid and butyric acid, which are compounds which are not part of the invention, do not make it possible to inhibit the expression of the iagA′-lacZ fusion in S. typhi;

that the sodium salts of valeric acid, caproic acid, oenanthic acid and pelargonic acid inhibit the expression of the iagA′-lacZ fusion in S. typhi as strongly as sodium caprylate;

that the presence of a hydroxyl radical at position 2 or 8 on the caprylic acid molecule (2-hydroxycaprylic acid and 8-hydroxycaprylic acid, respectively) does not modify the expression of the iagA′-lacZ fusion in S. typhi compared to that which is obtained with caprylic acid;

that the presence of an amino radical at position 2 or 8 on the caprylic acid molecule (2-aminocaprylic acid and 8-amionocaprylic acid, respectively) totally blocks the inhibitory activity of caprylic acid.

Consequently, this set of results demonstrates that the compounds of formula (I) in accordance with the invention make it possible to suppress, in vitro, the expression of the invasion genes in a Gram-negative bacterium such as Salmonella.

1. Use of an effective amount of at least one compound of formula (I) as follows:
R₁-COO-R₂ (I)
in which:
R₁ represents a C₄-C₉ saturated carbon-based chain, optionally substituted with one or more hydroxyl or amine functions, or with an aromatic ring;
R₂ represents a hydrogen atom, a monovalent alkali metal atom or an alkyl radical, it being understood that, when R₂ represents a hydrogen atom and R1 represents C₇ saturated carbon-based chain substituted with an amine function, then said amine function is not at position 2 or 8;
as an active principle, for preparing a pharmaceutical composition with a neutral pH intended to prevent Gram-negative bacterial infections, both in humans and in animals.

2. Use according to claim 1, characterized in that said Gram-negative bacterial infections are Salmonella infections.

3. Use according to claim 1 or 2, characterized in that the compounds of formula (I) are chosen from valeric acid, caproic acid, oenanthic acid, caprylic acid and pelargonic acid, their monohydroxylated derivatives, and also their salts and their esters.

4. Use according to any one of the preceding claims, characterized in that the compounds of formula (I) are chosen from those in which the R₁ radical represents a carbon-based chain substituted with a hydroxyl function, said hydroxyl function being at position 2 when the R₁ radical contains 4 to 6 carbon atoms and at position 2 or 8 when the R₁ radical contains 7 to 9 carbon atoms.

5. Use according to any one of the preceding claims, characterized in that the compounds of formula (I) are chosen from caprylic acid, its derivatives hydroxylated at position 2 or 8, their salts and their esters.

6. Use according to any one of the preceding claims, characterized in that the alkali metal atoms defined for the R₂ radical are chosen from sodium and potassium.

7. Use according to any one of the preceding claims, characterized in that the R₂ radical is a C₁-C₄ alkyl radical.

8. Use according to claim 6, characterized in that the C₁-C₄ alkyl radical defined for the R₂ radical is chosen from methyl, ethyl and butyl radicals.

9. Use of an effective amount of sodium caprylate, as an active principle, for preparing a pharmaceutical composition with a neutral pH intended to prevent Gram- negative bacterial infections, in particular Salmonella infections, in humans or in animals.

10. Use according to any one of the preceding claims, characterized in that the effective amount of said compounds of formula (I) corresponds to single doses of between 20 mM and 200 mM.

11. Use according to any one of the preceding claims, characterized in that the pharmaceutical composition is administered orally.

12. Use according to any one of the preceding claims, characterized in that the pharmaceutical composition is added to the drinking water and/or to the feed distributed to farm-stock animals.
